(19) United States
(12) Patent Application Publication
(10) Pub. No.: US 2021/0059621 A1
(43) Pub. Date: Mar. 4, 2021

(54) X-RAY IMAGE DIAGNOSTIC APPARATUS

(71) Applicant: Shimadzu Corporation, Kyoto (JP)

(72) Inventor: Keita OKUTANI, Kyoto (JP)

(21) Appl. No.: 16/679,898

(22) Filed: Nov. 11, 2019

(30) Foreign Application Priority Data
Aug. 27, 2019 (JP) 2019-154921

(51) Int. Cl. A61B 6/00

(52) U.S. Cl. CPC A61B 6/4452; A61B 6/4291; A61B 6/485

(57) ABSTRACT

An X-ray image diagnostic apparatus is provided with a top board, a movable X-ray source, an X-ray detection unit, a storage unit, an X-ray source drive unit, and a registered position operation part for performing an operation of moving the ray source to a registered position by the registered position drive unit. The X-ray image diagnostic apparatus is configured not to include an arbitrary position operation part for performing an operation of moving the X-ray source to an arbitrary position by the X-ray source drive unit.

### CROSS-REFERENCE TO RELATED APPLICATIONS

The related patent application number JP2019-154921, entitled X-ray image diagnostic apparatus, filed on Aug. 27, 2019, and invented by Keita Okutani upon which this patent application is based is hereby incorporated by reference.

### BACKGROUND OF THE INVENTION

### Field of the Invention

The present invention relates to an X-ray image diagnostic apparatus.

### Description of the Background Art

Conventionally, an X-ray image diagnostic apparatus for imaging a subject with X-rays is known. Such an X-ray image diagnostic apparatus is disclosed in, for example, Japanese Unexamined Patent Application Publication No. 2001-428.

In the above-described Japanese Unexamined Patent Application Publication No. 2001-428, a proximity operation type fluoroscopic imaging apparatus (X-ray image diagnostic apparatus) is disclosed in which a bed on which a patient is placed, an X-ray tube arranged below the bed, and an image system arranged above the bed are provided. This proximity operation type fluoroscopic imaging apparatus is a so-called under table tube type proximity operation type fluoroscopic imaging apparatus in which an X-ray tube is arranged below a bed.

Here, although not specifically described in the above-described Japanese Unexamined Patent Application Publication No. 2001-428, in the under table tube type proximity operation type fluoroscopic imaging apparatus in which an X-ray tube is arranged below the bed as described in Japanese Unexamined Patent Application Publication No 2001-428, in some cases, the X-ray source is configured to be able to move along a direction perpendicular to the top board. In this case, for example, in order to move the X-ray source to an appropriate position according to an examination of a subject, it is conceivable to provide an arbitrary position operation part for performing the operation of moving the X-ray source to an arbitrary position in the proximity operation type fluoroscopic imaging apparatus. However, in the case of providing an arbitrary position operation part for performing an operation to move the X-ray source to an arbitrary position, the operation of moving the X-ray source to an appropriate position using an arbitrary position operation part is likely to become complicated operations. For this reason, it is desired to easily move the X-ray source to an appropriate position.

### SUMMARY OF THE INVENTION

The present invention has been made to solve the aforementioned problems, and one object of the present invention is to provide an X-ray image diagnostic apparatus capable of easily moving an X-ray source to an appropriate position by preventing an operation of moving the X-ray source from becoming complicated.

In order to achieve the aforementioned object, an X-ray image diagnostic apparatus according to one aspect of the present invention includes:

a top board having a front surface and a back surface and configured to place a subject on the front surface;

a movable X-ray source provided on a side of the back surface of the top board and configured to emit X-rays;

an X-ray detection unit provided on a side of the front surface of the top board and configured to detect the X-rays emitted from the X-ray source and transmitted through the subject;

a storage unit configured to store a position of the X-ray source as a registered position;

an X-ray source drive unit configured to move the X-ray source; and

a registered position operation part configured to perform an operation of moving the X-ray source to the registered position by the X-ray source drive unit,

wherein an arbitrary position operation part configured to perform an operation of moving the X-ray source to an arbitrary position is not provided.

According to the present invention, the X-ray image diagnostic apparatus is provided with a registered position operation part configured to perform an operation of moving the X-ray source to a registered position by the X-ray source drive unit. With this, compared with the case in which an arbitrary position operation part is provided to move the X-ray source to an appropriate position, it is possible to suppress the operation of moving the X-ray source to an appropriate position (registered position) from becoming complicated. Further, by simply operating the registered position operation part, the X-ray source can be easily moved to the registered position which is a pre-registered appropriate position. As a result, an X-ray image diagnostic apparatus capable of easily moving an X-ray source to an appropriate position by suppressing the complicated operation of moving the X-ray source can be provided.

According to the present invention, the X-ray image diagnostic apparatus is not provided with an arbitrary position operation part configured to perform an operation of moving the X-ray source to an arbitrary position by the X-ray source drive unit. With this, unlike the case in which an arbitrary position operation part is provided separately and independently of the registered position operation part, since the number of operation parts for performing the operation of moving the X-ray source can be reduced, the complexity of the operation part of the X-ray image diagnostic apparatus can be suppressed accordingly.

### BRIEF DESCRIPTION OF THE DRAWINGS

FIG. 1 is a block diagram showing a configuration of an X-ray image diagnostic apparatus according to an embodiment.

FIG. 2 is a schematic perspective diagram showing an X-ray image diagnostic apparatus main body of an X-ray image diagnostic apparatus according to an embodiment.

FIG. 3A is a schematic diagram shown a state in which a top board of an X-ray image diagnostic apparatus according to an embodiment is horizontal.

FIG. 3B is a schematic diagram showing a state in which a top board of an X-ray image diagnostic apparatus according to an embodiment is vertical.

FIG. 4 is a schematic diagram showing an operation unit of an X-ray image diagnostic apparatus according to an embodiment.

FIG. 5 is a diagram for explaining a movement operation of an X-ray source and a top board of an X-ray image diagnostic apparatus according to an embodiment to a registered position.

FIG. 6 is a diagram for explaining selection and designation of an examination type indicating a registered position by a user of an X-ray image diagnostic apparatus according to an embodiment.

FIG. 7 is a diagram for explaining a movement operation of an X-ray source and a top board to a registered position when performing a chest examination with an X-ray image diagnostic apparatus according to one embodiment.

FIG. 8 is a diagram for explaining a movement operation of an X-ray source and a top board to a registered position when performing a swallowing examination with an X-ray image diagnostic apparatus according to an embodiment.

FIG. 9 is a diagram for explaining registered position movement processing of an X-ray image diagnostic apparatus according to an embodiment.

### DESCRIPTION OF PREFERRED EMBODIMENTS

Hereinafter, embodiments of the present invention will be described with reference to the attached drawings.

### (Configuration of X-Ray Image Diagnostic Apparatus)

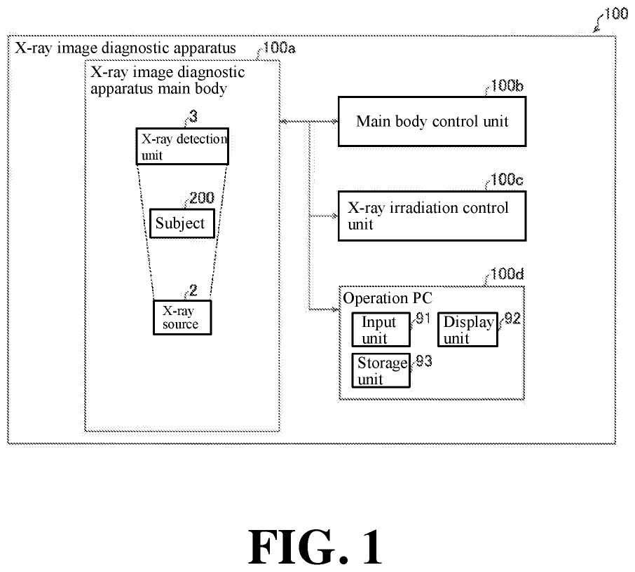

With reference to FIG. 1 to FIG. 8, the configuration of an X-ray image diagnostic apparatus 100 according to an embodiment will be described.

As shown in FIG. 1, the X-ray image diagnostic apparatus 100 is an apparatus for imaging a subject 200 that is a human body with X-rays. The X-ray image diagnostic apparatus 100 is configured to generate an image of an inside of the subject 200 based on an imaging result of the subject 200 by X-rays. In this embodiment, the X-ray image diagnostic apparatus 100 is an X-ray fluoroscopic imaging apparatus (so-called X-ray television apparatus) capable of generating the image of the inside of the subject 200 in real time.

The X-ray image diagnostic apparatus 100 is provided with an X-ray image diagnostic apparatus main body 100a (hereinafter simply referred to as “apparatus main body 100a”), a main body control unit 100b, an X-ray irradiation control unit 100c, and an operation PC (personal computer) 100d. The apparatus main body 100a, the main body control unit 100b, the X-ray irradiation control unit 100c, and the operation PC 100d are communicably connected to each other. The apparatus main body 100a is configured to image the subject 200 with X-rays. The detailed configuration of the apparatus main body 100a will be described later. The main body control unit 100b is configured to control the operation of the apparatus main body 100a. The main body control unit 100b includes, for example, a processor, such as a CPU, and a memory that stores information. The X-ray irradiation control unit 100c is configured to control X-ray irradiation of the apparatus main body 100a. The X-ray irradiation control unit 100c includes, for example, a processor, such as a CPU, and a memory that stores information.

The operation PC 100d is a personal computer for a user operation. The operation PC 100d includes an input unit 91, a display unit 92, and a storage unit 93. The input unit 91 is an interface, such as mouse and a keyboard, for a user to perform an input operation. The display unit 92 includes, for example, a liquid crystal monitor, and is configured to display information, such as, e.g., the image of the inside of the subject 200 acquired by the apparatus main body 100a. The storage unit 93 includes a storage medium, such as, e.g., a flash memory, and is configured to store information. In this embodiment, the storage unit 93 is storing registered positions which will be described later. Note that the registered position and the control operation using the registered position will be described later.

(X-Ray Image Diagnostic Apparatus Main Body)

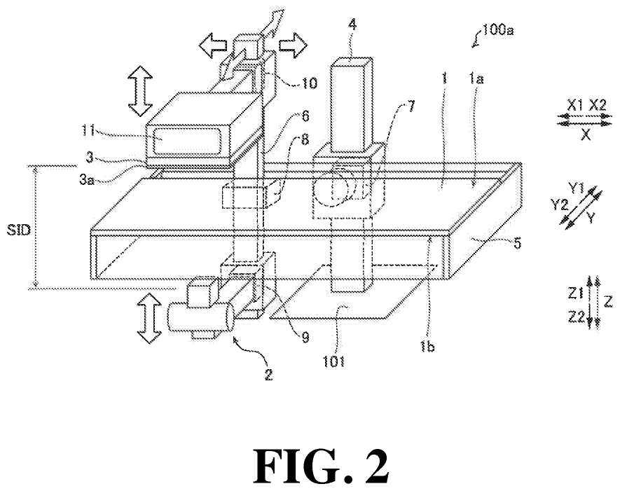

As shown in FIG. 2, the apparatus main body 100a is provided with, as imaging mechanisms, a top board 1, an X-ray source 2, and an X-ray detection unit 3. The top board 1 has a front surface 1a that is a surface on which a subject 200 that is an imaging target (examination target) is placed, and a back surface 1b that is a surface opposite to the front surface 1a. The top board 1 has a substantially rectangular shape when viewed from a direction (in FIG. 2, in the Z-direction) substantially perpendicular to the front surface 1a and the back surface 1b. In FIG. 2, the top board 1 has a longitudinal direction in the X-direction and a short direction in the Y-direction.

The X-ray source 2 generates X-rays and emits the X-rays to a subject 200. The X-ray source 2 includes, for example, an X-ray tube (not shown) for generating X-rays and a collimator (not shown) for adjusting the irradiation range of the X-rays generated by the X-ray tube.

The X-ray detection unit 3 detects X-rays irradiated from the X-ray source 2 and transmitted through the subject 200. The X-ray detection unit 3 has, for example, an FPD (Flat Panel Detector). The X-ray detection unit 3 transmits a detection signal which is an electric signal corresponding to the detected X-ray to the image processing unit. The image processing unit generates the image of the inside of the subject 200 based on a detection signal received from the X-ray detection unit 3. Further, the X-ray detection unit 3 is configured so that a grid 3a can be detachably attached. The grid 3a is a filter member for removing scattered radiation (secondary X-rays) generated when X-rays transmit through the subject 200. By using the grid 3a, the image of the inside of the subject 200 made more clear by removal of the scattered radiation can be obtained. Further, as the grid 3a, for example, a focusing grid can be used. In FIG. 2, a state in which the grid 3a is attached to the X-ray detection unit 3 is illustrated.

In the apparatus main body 100a, the X-ray source 2 is provided on the side of the back surface 1b of the top board 1 with respect to the top board 1 (in FIG. 2, on the Z2-direction side), and the X-ray detection unit 3 is provided on the side of the front surface 1a of the top board 1 with respect to the top board 1 (in FIG. 2, on the Z1-direction side). The X-ray image diagnostic apparatus 100 is a so-called under tube type X-ray image diagnostic apparatus. The X-ray source 2 and the X-ray detection unit 3 are provided so as to face each other with the top board 1 interposed therebetween.

Further, the apparatus main body 100a is provided, as support mechanisms, a leg portion 4, a main frame 5, and a support column 6. The leg portion 4 is a support unit that supports the entire apparatus main body 100a. The leg portion 4 is fixedly provided on the installation surface 101 which is the floor surface of the room where the apparatus main body 100a is installed. The leg portion 4 is provided so as to support the main frame 5 in a movable manner. The main frame 5 is a support unit that supports the top board 1 and the support column 6. The main frame 5 is provided so as to fixedly support the top board 1 and movably support the support column 6. The support column 6 is a support unit for supporting the X-ray source 2 and the X-ray detection unit 3. The support column 6 is provided so as to support the X-ray source 2 in a movable manner on the side of the back surface 1b (in FIG. 2, on the Z2-direction side) of the top board 1 with respect to the top board 1, and is also provided so as to support the X-ray detection unit 3 in a movable manner on the side of the front surface 1a (in FIG. 2, on the Z1-direction side) of the top board 1 with respect to the top board 1.

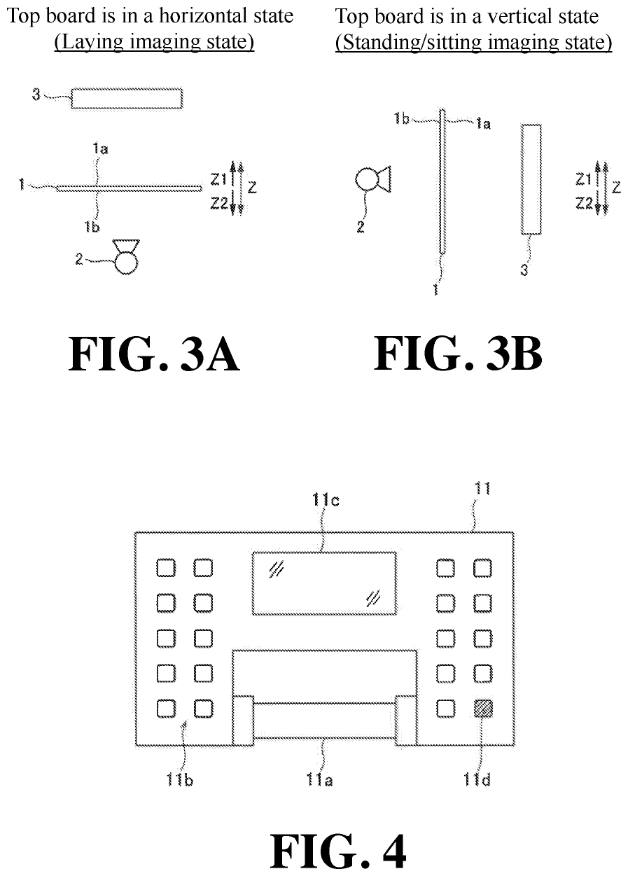

Further, the apparatus main body 100a is provided, as drive mechanisms, a top board drive unit 7, a support column drive unit 8, an X-ray source drive unit 9, and a detection unit drive unit 10. The top board drive unit 7 includes a drive motor, and is configured to rotationally move the top board 1 between a horizontal state (see FIG. 3A) and a vertical state (see FIG. 3B). Specifically, the top board drive unit 7 is configured to integrally rotationally move the top board 1 and the support column 6 supported by the main frame 5 and the X-ray source 2 and the X-ray detection unit 3 supported by the support column 6 by rotationally moving the main frame 5. The board drive unit 7 is provided to the leg portion 4.

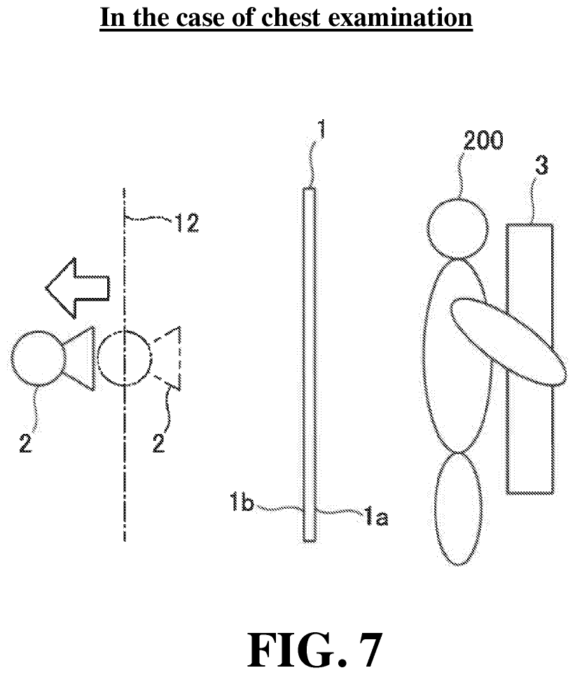

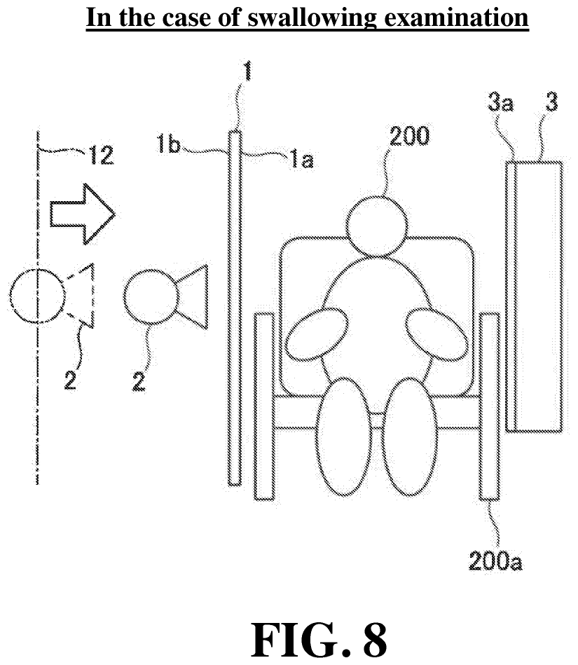

As shown in FIG. 3A, when the top board 1 is in a horizontal state, imaging of the subject 200 in a lying posture is performed. That is, when the top board 1 is in a horizontal state, imaging of the subject 200 by the X-ray source 2 and the X-ray detection unit 3 is performed in a state in which the subject 200 is placed on the front surface 1a of the top board 1 in a lying posture. Further, as shown in FIG. 3B, when the top board 1 is in a vertical state, imaging of the subject 200 is performed in a standing or sitting posture (see FIG. 7 and FIG. 8). That is, when the top board 1 is in a vertical state, in a state in which the subject 200 is not placed on the front surface 1a of the top board 1, imaging of the subject 200 who is in the standing posture or sitting posture is performed by the X-ray source 2 and the X-ray detection unit 3.

As shown in FIG. 2, the support column drive unit 8 includes a drive motor, and is configured to linearly move the support column 6 in a direction along the longitudinal direction of the top board 1 (in FIG. 2, in the X-direction) and in the direction along the short direction of the top board 1 (in FIG. 2, in the Y-direction). With this, the support column drive unit 8 is configured to linearly integrally move the X-ray source 2 and the X-ray detection unit 3 supported by the support column 6 while maintaining the facing state. The support column drive unit 8 is provided to the main frame 5.

The X-ray source drive unit 9 includes a drive motor, and is configured to linearly move the X-ray source 2 along a direction perpendicular to the top board 1 (in FIG. 2, in the Z-direction). That is, the X-ray source drive unit 9 is configured to linearly move the X-ray source 2 in a direction getting close to the top board 1 (in FIG. 2, in the Z1-direction) and in a direction getting away from the top board 1 (in FIG. 2, in the Z2-direction). The X-ray source drive unit 9 is provided on one side of the support column 6 (in FIG. 2, on the Z2-direction side). The X-ray source 2 is configured to be able to be linearly moved by the X-ray source drive unit 9 in any of the state in which the top board 1 is horizontal (see FIG. 3A), the state in which the top board 1 is vertical (see FIG. 3B), and the state in which the top board 1 is being rotated.

The detection unit drive unit 10 is configured to linearly move the X-ray detection unit 3 along a direction perpendicular to the top board 1. That is, the detection unit drive unit 10 is configured to linearly move the X-ray detection unit 3 in a direction getting close to the top board 1 and in a direction getting away from the top board 1. The detection unit drive unit 10 is provided on the other side of the support column 6 (in FIG. 2, on the Z1-direction side). The X-ray source 2 and the X-ray detection unit 3 are configured to be movable independently of each other in a direction perpendicular to the top board 1. With this, by moving at least one of the X-ray source 2 and the X-ray detection unit 3, the SID (Source Image Distance) that is the distance between the X-ray source 2 and the X-ray detection unit 3 can be adjusted.

Further, the apparatus main body 100a is provided with an operation unit 11 (operation panel) for performing an operation of moving, e.g., the X-ray source 2 and the X-ray detection unit 3. The operation unit 11 is provided in the vicinity of the X-ray detection unit 3. The user can perform an operation of moving, e.g., the X-ray source 2 and the X-ray detection unit 3 at a position close to the subject 200 by using the operation unit 11. The X-ray image diagnostic apparatus 100 is a so-called close operation type X-ray image diagnostic apparatus.

As shown in FIG. 4, the operation unit 11 is provided with a detection unit operation part 11a for performing a movement operation of the X-ray detection unit 3 by the detection unit drive unit 10. The user can perform an operation of linearly moving the X-ray detection unit 3 along a direction perpendicular to the top board 1 using the detection unit operation part 11a. The detection unit operation part 11a is an operation handle to be held and operated by a user. The detection unit operation part 11a as an operation handle has a rod shape. The detection unit operation part 11a is also an operation part for performing the operation of moving the support column 6 by the support column drive unit 8. The user can linearly and integrally move, using the detection unit operation part 11a, the X-ray source 2 and the X-ray detection unit 3 supported by the support column 6 while maintaining the facing state in a direction along the longitudinal direction of the top board 1 and in a direction along the short direction of the top board 1.

Further, the operation unit 11 is provided with an operation switch group 11b and a liquid crystal panel 11c. The operation switch group 11b is composed of a plurality of push button switches for performing various operations. The operation switch group 11b includes, for example, an operation switch for operating the movement of the top board 1, an operation switch for inputting setting values related to X-ray irradiation, and the like. Further, the liquid crystal panel 11c is configured to display information necessary for the examination (imaging) of the subject 200. The liquid crystal panel 11c displays information, such as, e.g., the examination type of the subject 200 and the position of each part (the top board 1, the X-ray source 2, the X-ray detection unit 3, etc.).

Here, in this embodiment, the operation unit 11 is provided with a registered position operation part 11d for performing the operation of moving the X-ray source 2 by the X-ray source drive unit 9 to a registered position stored in the storage unit 93 (see FIG. 1) of the operation PC 100d. On the other hand, the X-ray image diagnostic apparatus 100 of this embodiment is configured not to include an arbitrary position operation part (joystick or the like) for moving the X-ray source 2 to an arbitrary position by the X-ray source drive unit 9. The storage unit 93 is configured to be able to store the position of the X-ray source 2 and the angle of the top board 1 as a registered position. That is, the registered position operation part 11d is also an operation part for performing the operation of moving the top board 1 by the top board drive unit 7. Note that the position of the X-ray source 2 specifically means the position of the X-ray source 2 in a direction along a direction perpendicular to the top board 1. Further note that the angle of the top board 1 specifically means the rotation angle of the top board 1 by the top board drive unit 7.

The registered position operation part 11d is a push button type operation switch. The registered position operation part 11d, which is an operation switch, is composed of one operation switch. The registered position operation part 11d is provided in the vicinity of the detection unit operation part 11a.

(Movement Operation to Registered Position)

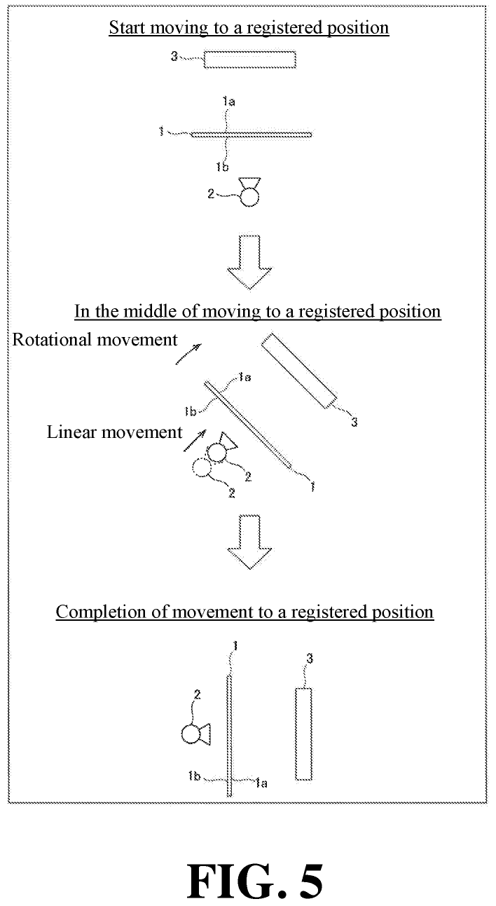

As shown in FIG. 5, when the registered position operation part 11d is operated, the main body control unit 100b is configured to perform control of moving the X-ray source 2 and the top board 1 to a registered position by linearly moving the X-ray source 2 along a direction perpendicular to the top board 1 by the X-ray source drive unit 9 and rotationally moving the top board 1 by the top board drive unit 7. Further, when the X-ray source 2 and the top board 1 are moved to a registered position, the main body control unit 100b is configured to perform the linear movement of the X-ray source 2 and the rotational movement of the top board 1 in parallel. That is, the X-ray source 2 is linearly moved while the top board 1 is being rotated.

The main body control unit 100b is configured to perform control of moving the X-ray source 2 to a registered position by the X-ray source drive unit 9 and also moving the top board 1 to a registered position by the top board drive unit 7 when the registered position operation part 11d is being operated. That is, the main body control unit 100b is configured to not perform the movement control of the X-ray source 2 by the X-ray source drive unit 9 and the movement control of the top board 1 by the top board drive unit 7 when the operation of the registered position operation part 11d is not recognized. For example, when the operation of registered position operation part 11d by the user is not recognized during the movement of the X-ray source 2 and the top board 1 to a registered position, the movement of the X-ray source 2 by the X-ray source drive unit 9 and the movement of the top board 1 by the board drive unit 7 are stopped.

The main body control unit 100b is configured to stop moving the X-ray source 2 by the X-ray source drive unit 9 and moving the top board 1 by the top board drive unit 7 also when the X-ray source 2 and the top board 1 have reached the registered position. Whether the X-ray source 2 has reached the registered position can be determined based on, for example, an output value of a position sensor (encoder or the like) of a drive motor of the X-ray source drive unit 9. Similarly, whether the top board 1 has reached a registered position can be determined based on, for example, an output value of a position sensor (encoder or the like) of a drive motor of the top board drive unit 7.

(Selection and Designation of Registered Position)

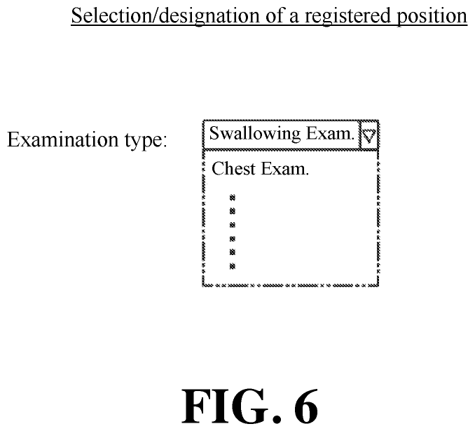

In this embodiment, as shown in FIG. 6, the storage unit 93 is configured to be able to store a plurality of registered positions so as to correspond to a plurality of types of examinations (imaging) of the subject 200. That is, the storage unit 93 is configured to be able to store different registered positions for each examination of the subject 200. In the example shown in FIG. 6, as the registered positions, registered positions, such as a registered position that is an imaging position at the time of a swallowing examination of the subject 200 and a registered position that is an imaging position at the time of a chest examination of the subject 200 are stored in the storage unit 93. Storing (registering) the registered position in the storage unit 93 may be performed before shipping the X-ray image diagnostic apparatus 100 or may be performed by an input operation of a user. That is, the registered position may be a predetermined fixed position or a position designated by the user.

The user can designate a registered position for performing the examination out of a plurality of registered positions stored in the storage unit 93 using the operation PC 100d. When designating a registered position, the operation PC 100d displays a setting screen for designating a registered position for performing an examination among the plurality of registered positions on the display unit 92. In the setting screen shown in FIG. 6, the examination type is displayed on the display unit 92 as the information for specifying a registered position. The user performs an operation of designating a registered position for performing an examination among a plurality of registered positions by using the input unit 91. The operation PC100d performs control of transmitting the registered position designated by the user using the input unit 91 to the main body control unit 100b.

When the main body control unit 100b receives a registered position designated by the user from the operation PC 100d, the main body control unit 100b performs control of making the operation of the registered position operation part 11d effective. When the registered position operation part 11d is operated, the main body control unit 100b is configured to perform control of moving the X-ray source 2 by the X-ray source drive unit 9 to the registered position designated by the user and moving the top board 1 by the top board drive unit 7 among the plurality of registered positions stored in the storage unit 93. Note that when the main body control unit 100b has not received the registered position designated by the user from the operation PC 100d, since the operation of the registered position operation part 11d is invalid, even when the registered position operation part 11d is operated by the user, the movement control of the X-ray source 2 and the top board 1 to the registered position is not performed.

(Registered Position in Case of Chest Examination)

With reference to FIG. 7, the registered position in the case of a chest examination of a subject 200 will be described.

As shown in FIG. 7, in the chest examination of the subject 200, increasing the SID is effective for imaging the subject 200. Therefore, in order to increase the SID, as the position of the X-ray source 2 as a registered position, a position shifted in a direction getting away from the top board 1 along a direction perpendicular to the top board 1 from the reference position 12 (indicated by the a dot-and-dash line) (that is, in a direction getting away from the X-ray detection unit 3) is stored in the storage unit 93. Further, the chest examination of the subject 200 is imaging of the subject 200 in a standing posture. For this reason, as the angle of the top board 1 as a registered position, the angle of the top board 1 in the vertical state (that is, 90 degrees with respect to the horizontal plane) is stored in the storage unit 93.

In this case, when the registered position operation part 11d is pressed, the X-ray source 2 is linearly moved to a position shifted in a direction getting away from the top board 1 by the X-ray source drive unit 9 along a direction perpendicular to the top board 1 from the reference position 12. In parallel to the movement of the X-ray source 2, the top board 1 is rotationally moved by the top board drive unit 7 so as to become the angle of the top board 1 in the vertical state. When the top board 1 is being rotated, the X-ray source 2 and the X-ray detection unit 3 are also rotationally moved together with the top board 1. At this time, since the registered position does not include the position of the X-ray detection unit 3, the X-ray detection unit 3 does not move along the direction perpendicular to the top board 1 by the detection unit drive unit 10. Note that, after the X-ray source 2 and the top board 1 have reached the registered position and stopped, the X-ray detection unit 3 may be further moved using the detection unit operation part 11a of the operation unit 11.

(Registered Position in the Case of Swallowing Examination)

With reference to FIG. 8, the registered position in the case of a swallowing examination of a subject 200 will be described.

As shown in FIG. 8, in a swallowing examination of a subject 200, there may be a case in which the subject 200 is seated on a wheelchair 200a, so that the distance between the top board 1 and the X-ray detection unit 3 may increase. In this case, the SID may sometimes become larger than the focusing distance of the grid 3a that is a focusing grid. For this reason, in order to keep the SID within the focusing distance of the grid 3a, as the position of X-ray source 2 as a registered position, a position shifted in a direction getting close to the top board 1 along the direction perpendicular to the top board 1 (that is, a direction getting close to the X-ray detection unit 3) from the reference position 12 is stored in the storage unit 93. Further, the swallowing examination of the subject 200 is imaging of the subject 200 in the sitting posture or standing posture. For this reason, as the angle of the top board 1 as a registered position, the angle of the top board 1 in a vertical state is stored in the storage unit 93.

In this case, when the registered position operation part 11d is pressed, the X-ray source 2 is linearly moved to a position shifted in a direction getting close to the top board 1 by the X-ray source drive unit 9 along a direction perpendicular to the top board 1 from the reference position 12. In parallel to the movement of the X-ray source 2, the top board 1 is rotationally moved by the top board drive unit 7 so as to become the angle of the top board 1 in the vertical state. When the top board 1 is rotationally moved, the X-ray source 2 and the X-ray detection unit 3 are also rotationally moved together with the top board 1. At this time, since the registered position does not include the position of the X-ray detection unit 3, the X-ray detection unit 3 does not move along the direction perpendicular to the top board 1 by the detection unit drive unit 10. Note that, after the X-ray source 2 and the top board 1 have reached the registered position and stopped, the X-ray detection unit 3 may be further moved using the detection unit operation part 11a of the operation unit 11.

(Registered Position Movement Processing)

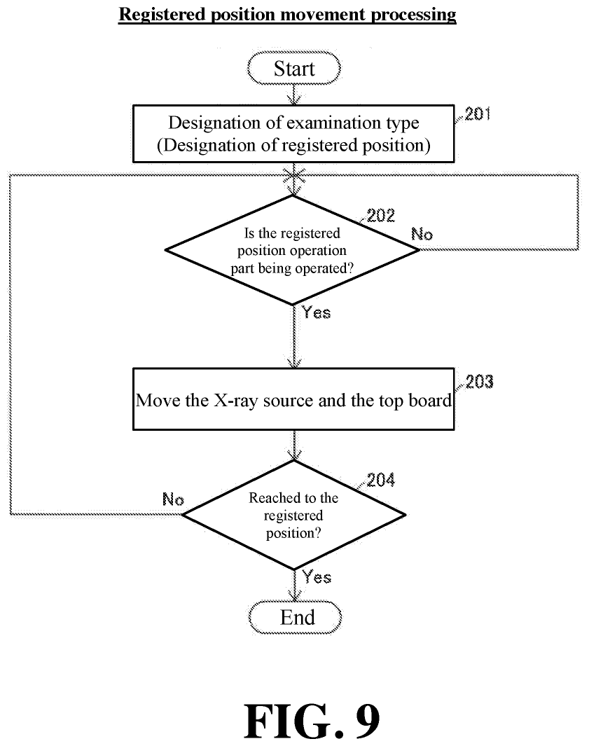

Next, with reference to FIG. 9, the registered position movement processing by the X-ray image diagnostic apparatus 100 of this embodiment will be described based on a flowchart. Each processing of the flowchart is performed by the main body control unit 100b.

As shown in FIG. 9, first, in Step 201, a registered position (examination type) is designated from among a plurality of registered positions stored in the storage unit 93. Specifically, in Step 201, the registered position designated by the user is acquired.

In Step 202, it is determined whether the registered position operation part 11d is being operated. When it is determined that the registered position operation part 11d is not being operated, the processing of Step 202 is repeated. When it is determined that the registered position operation part 11d is being operated, the processing proceeds to Step 203.

In Step 203, the movement of the X-ray source 2 to the registered position by the X-ray source drive unit 9 and the movement of the top board 1 to the registered position by the top board drive unit 7 are performed.

In Step 204, it is determined whether the X-ray source 2 and the top board 1 have reached the registered position. If it is determined that the X-ray source 2 and the top board 1 have not reached the registered position, the processing returns to Step 202. Then, the processing of Step 202 and the processing of Step 203 are repeated. If it is determined that the X-ray source 2 and the top board 1 have reached the registered position, the registered position moving processing is terminated.

### Effect of This Embodiment

In this embodiment, the following effects can be obtained.

In this embodiment, as described above, the X-ray image diagnostic apparatus 100 is provided with the registered position operation part 11d for the operation of moving the X-ray source 2 to the registered position by the X-ray source drive unit 9. With this, compared with the case in which an arbitrary position operation part is provided in order to move the X-ray source 2 to an appropriate position, it is possible to prevent the operation of moving the X-ray source 2 to an appropriate position (registered position) from becoming complicated. Further, by simply operating the registered position operation part 11d, the X-ray source 2 can be easily moved to the registered position which is a pre-registered appropriate position. As a result, an X-ray image diagnostic apparatus 100 capable of easily moving the X-ray source 2 to an appropriate position by suppressing the complicated operation of moving the X-ray source 2 can be provided. Further, according to this embodiment, the X-ray image diagnostic apparatus 100 is not provided with an arbitrary position operation unit for performing an operation of moving the X-ray source 2 to an arbitrary position by the X-ray source drive unit 9. With this, unlike the case in which an arbitrary position operation part is provided separately and independently of the registered position operation part 11d, since the number of operation parts for performing the operation of moving the X-ray source 2 can be reduced, the complexity of the operation part of the X-ray image diagnostic apparatus 100 can be suppressed accordingly.

Further, in this embodiment, as described above, the X-ray image diagnostic apparatus 100 is configured to include the top board drive unit 7 for rotationally moving the top board 1 between a horizontal state and a vertical state. Further, the X-ray source 2 is configured to be able to be moved by the X-ray source drive unit 9 in any of the state in which the top board 1 is horizontal, the state in which the top board 1 is vertical, and the state in which the top board 1 is being rotated. With this, even in any of the state in which the top board 1 is horizontal and the state in which the top board 1 is vertical, since the X-ray source 2 can be moved by the X-ray source drive unit 9, the X-ray source 2 can be appropriately moved by the X-ray source drive unit 9. Therefore, the X-ray source 2 can be appropriately moved by the X-ray source drive unit 9 according to the state of the top board 1.

Further, in this embodiment, as described above, the storage unit 93 is configured to be able to store the position of the X-ray source 2 and the angle of the top board 1 as a registered position. Further, the X-ray image diagnostic apparatus 100 is configured to move the X-ray source 2 and the top board 1 to a registered position by linearly moving the X-ray source 2 along a direction perpendicular to the top board 1 by the X-ray source drive unit 9 and rotationally moving the top board 1 by the top board drive unit 7 when the registered position operation part 11d is operated. With this, by simply operating the registered position operation part 11d, both the X-ray source 2 and the top board 1 can be easily moved to a registered position which is a pre-registered appropriate position. As a result, unlike the case in which the X-ray source 2 and the top board 1 need to be moved separately and independently, the operation of moving the X-ray source 2 and the top board 1 to the registered position can be further suppressed from becoming complicated.

Further, in this embodiment, as described above, the X-ray image diagnostic apparatus 100 is configured such that when the X-ray source 2 and the top board 1 are moved to a registered position, the linear movement of the X-ray source 2 and the rotational movement of the top board 1 are performed in parallel. With this, compared with the case in which the linear movement of the X-ray source 2 and the rotational movement of the top board 1 are not performed in parallel, the time required to move the X-ray source 2 and the top board 1 to the registered position can be reduced.

Further, in this embodiment, as described above, the X-ray image diagnostic apparatus 100 is configured to move the X-ray source 2 to the registered position by the X-ray source drive unit 9 while the registered position operation part 11d is being operated. With this, the user can stop the movement of the X-ray source 2 to a registered position by simply stopping the operation of the registered position operation part 11d. As a result, when it is desired to stop the movement of the X-ray source 2 to the registered position, the movement of the X-ray source 2 to the registered position can be stopped easily and quickly.

Further, in this embodiment, as described above, the storage unit 93 is configured to be able to store a plurality of registered positions so as to correspond to a plurality of types of examinations of the subject 200. Further, the X-ray image diagnostic apparatus 100 is configured to move the X-ray source 2 to a registered position designated among a plurality of registered positions by the X-ray source drive unit 9 when the registered position operation part 11d is operated. With this, the user can easily move the X-ray source 2 to a registered position corresponding to the examination to be carried out by simply designating the registered position to be carried out among the plurality of registered positions and operating the registered position operation part 11d.

Further, in this embodiment, as described above, the X-ray image diagnostic apparatus 100 is configured to include the detection unit drive unit 10 for moving the X-ray detection unit 3, a detection unit operation part 11a for operating the movement of the X-ray detection unit 3 by the detection unit drive unit 10, and the operation unit 11 provided with the detection unit operation part 11a and the registered position operation part 11d. With this, compared with the case in which the detection unit operation part 11a and the registered position operation part 11d are provided in separate operation units, the operation of moving the X-ray detection unit 3 using the detection unit operation part 11a and the operation of moving the X-ray source 2 using the registered position operation part 11d can be easily performed using the operation unit 11 provided with both the detection unit operation part 11a and the registered position operation part 11d.

In this embodiment, as described above, the detection unit operation part 11a is configured to be an operation handle. Further, the registered position operation part 11d is configured to be an operation switch. With this, the detection unit operation part 11a and the registered position operation part 11d can be simply configured.

In this embodiment, as described above, the registered position operation part 11d is configured by one operation switch. With this, compared with the case in which the registered position operation part 11d is configured by two or more operation switches, since the number of registered position operation part 11d can be reduced, the structure of the operation part of the X-ray image diagnostic apparatus 100 can be further suppressed from becoming complicated. Further, unlike the case in which the registered position operation part 11d is composed of two or more operation switches, it is possible to suppress occurrence of erroneous operations of the registered position operation part 11d.

### Modified Embodiment

It should be understood that the embodiments disclosed here are examples in all respects and are not restrictive. The scope of the present invention is shown by the scope of the claims rather than the descriptions of the embodiments described above, and includes all changes (modifications) within the meaning of equivalent and the scope of claims.

For example, in the above-described embodiment, an example is shown in which the X-ray image diagnostic apparatus is an X-ray fluoroscopic imaging apparatus that performs X-ray fluoroscopic imaging, but the present invention is not limited to this. In the present invention, the X-ray image diagnostic apparatus may be an X-ray imaging apparatus that performs X-ray imaging (so-called general imaging) other than X-ray fluoroscopic imaging.

In the above-described embodiment, an example is shown in which the X-ray image diagnostic apparatus is a proximity operation type X-ray image diagnostic apparatus, but the present invention is not limited to this. In the present invention, the X-ray image diagnostic apparatus may be a remote control type X-ray image diagnostic apparatus.

In the above-described embodiment, an example is shown in which the position of the X-ray source and the angle of the top board are stored in the storage unit as a registered position, however, the present invention is not limited to this. In the present invention, as long as the X-ray source position is stored in the storage unit as a registered position, the angle of the top board does not necessarily have to be stored in the storage unit. Further, as the registered position, any position other than the angle of the top board (for example, the position of the X-ray detection unit) may be stored in the storage unit.

Further, in the above-described embodiment, an example is shown in which the storage unit for storing the registered position is provided in the operation PC, the present invention is not limited to this. In the present invention, the storage unit that stores the registered position may be provided in a place other than the operation PC (for example, apparatus main body).

Further, in the above embodiment, an example is shown in which the X-ray image diagnostic apparatus is configured to perform the linear movement of the X-ray source and the rotational movement of the top board in parallel when moving the X-ray source and the top board to the registered position. However, the present invention is not limited to this. In the present invention, when the X-ray image diagnostic apparatus moves the X-ray source and the top board to the registered position, it may be configured such that the linear movement of the X-ray source and the rotational movement of the top board are performed independently of each other.

Further, in the above embodiment, an example is shown in which a plurality of registered positions is stored in the storage unit, but the present invention is not limited to this. In the present invention, only one registered position may be stored in the storage unit.

In the above embodiment, an example is shown in which the registered position operation part is composed of one operation switch, but the present invention is not limited to this. In the present invention, the registered position operation part may be composed of two or more operation switches. In this case, different registered positions may be assigned to the plurality of operation switches.

### Aspects

It is understood by those skilled in the art that the above-described exemplary embodiments are specific examples of the following aspects.

(Item 1)

An X-ray image diagnostic apparatus comprising:

a top board having a front surface and a back surface and configured to place a subject on the front surface;

a movable X-ray source provided on a side of the back surface of the top board and configured to emit X-rays;

an X-ray detection unit provided on a side of the front surface of the top board and configured to detect the X-rays emitted from the X-ray source and transmitted through the subject;

a storage unit configured to store a position of the X-ray source as a registered position;

an X-ray source drive unit configured to linearly move the X-ray source along a direction perpendicular to the top board; and

a registered position operation part configured to perform an operation of moving the X-ray source to the registered position by the X-ray source drive unit,

wherein an arbitrary position operation part configured to perform an operation of moving the X-ray source to an arbitrary position is not provided.

(Item 2)

The X-ray image diagnostic apparatus as recited in the aforementioned Item 1, further comprising:

a top board drive unit configured to rotationally move the top board between a horizontal state and a vertical state,

wherein the X-ray source is configured to be able to be moved by the X-ray source drive unit in both a state in which the top board is horizontal and a state in which the top board is vertical.

(Item 3)

The X-ray image diagnostic apparatus as recited in the aforementioned Item 2,

wherein the storage unit is configured to store a position of the X-ray source and an angle of the top board as the registered position, and

wherein it is configured such that when the registered position operation part is operated, the X-ray source and the top board are moved to the registered position by linearly moving the X-ray source along the direction perpendicular to the top board by the X-ray source drive unit and rotationally moving the top board by the top board drive unit.

(Item 4)

The X-ray image diagnostic apparatus as recited in the aforementioned Item 3,

wherein it is configured such that when the X-ray source and the top board are moved to the registered position, a linear movement of the X-ray source and a rotational movement of the top board are performed in parallel.

(Item 5)

The X-ray image diagnostic apparatus as recited in the aforementioned any one of Items 1 to 4,

wherein it is configured such that while the registered position operation part is being operated, the X-ray source is moved to the registered position by the X-ray source drive unit.

(Item 6)

The X-ray image diagnostic apparatus as recited in any one of the aforementioned Items 1 to 5,

wherein the storage unit is configured to be able to store a plurality of registered positions so as to correspond to a plurality of types of examinations of the subject, and

wherein it is configured such that when the registered position operation part is operated, the X-ray source is moved by the X-ray source drive unit to a registered position designated by a user among the plurality of registered positions

(Item 7)

The X-ray image diagnostic apparatus as recited in any one of the aforementioned Items 1 to 6, further comprising:

a detection unit drive unit configured to move the X-ray detection unit;

a detection unit operation part configured to perform an operation of moving the X-ray detection unit by the detection unit drive unit; and

an operation unit provided with the detection unit operation part and the registered position operation part.

(Item 8)

The X-ray image diagnostic apparatus as recited in the aforementioned Item 7,

wherein the detection unit operation part is an operation handle, and

wherein the registered position operation part is an operation switch.

(Item 9)

The X-ray image diagnostic apparatus as recited in the aforementioned Item 8,

wherein the registered position operation part is composed of one operation switch.

1. An X-ray image diagnostic apparatus comprising:
a top board having a front surface and a back surface and configured to place a subject on the front surface;
a movable X-ray source provided on a side of the back surface of the top board and configured to emit X-rays;
an X-ray detection unit provided on a side of the front surface of the top board and configured to detect the X-rays emitted from the X-ray source and transmitted through the subject;
a storage unit configured to store a position of the X-ray source as a registered position;
an X-ray source drive unit configured to linearly move the X-ray source along a direction perpendicular to the top board; and
a registered position operation part configured to perform an operation of moving the X-ray source to the registered position by the X-ray source drive unit,
wherein an arbitrary position operation part configured to perform an operation of moving the X-ray source to an arbitrary position is not provided.

2. The X-ray image diagnostic apparatus as recited in claim 1, further comprising:
a top board drive unit configured to rotationally move the top board between a horizontal state and a vertical state,
wherein the X-ray source is configured to be able to be moved by the X-ray source drive unit in both a state in which the top board is horizontal and a state in which the top board is vertical.

3. The X-ray image diagnostic apparatus as recited in claim 2,
wherein the storage unit is configured to store a position of the X-ray source and an angle of the top board as the registered position, and
wherein it is configured such that when the registered position operation part is operated, the X-ray source and the top board are moved to the registered position by linearly moving the X-ray source along the direction perpendicular to the top board by the X-ray source drive unit and rotationally moving the top board by the top board drive unit.

4. The X-ray image diagnostic apparatus as recited in claim 3,
wherein it is configured such that when the X-ray source and the top board are moved to the registered position, a linear movement of the X-ray source and a rotational movement of the top board are performed in parallel.

5. The X-ray image diagnostic apparatus as recited in claim 1,
wherein it is configured such that while the registered position operation part is being operated, the X-ray source is moved to the registered position by the X-ray source drive unit.

6. The X-ray image diagnostic apparatus as recited in claim 1,
wherein the storage unit is configured to be able to store a plurality of registered positions so as to correspond to a plurality of types of examinations of the subject, and
wherein it is configured such that when the registered position operation part is operated, the X-ray source is moved by the X-ray source drive unit to a registered position designated by a user among the plurality of registered positions.

7. The X-ray image diagnostic apparatus as recited in claim 1, further comprising:
a detection unit drive unit configured to move the X-ray detection unit;
a detection unit operation part configured to perform an operation of moving the X-ray detection unit by the detection unit drive unit; and
an operation unit provided with the detection unit operation part and the registered position operation part.

8. The X-ray image diagnostic apparatus as recited in claim 7,
wherein the detection unit operation part is an operation handle, and
wherein the registered position operation part is an operation switch.

9. The X-ray image diagnostic apparatus as recited in claim 8,
wherein the registered position operation part is composed of one operation switch.
